(19) United States
(12) Patent Application Publication
(10) Pub. No.: US 2020/0256860 A1
(43) Pub. Date: Aug. 13, 2020

(54) PRINTABLE HYDROGELS FOR BIOMOLECULE IMMOBILIZATION AND STABILIZATION

(71) Applicant: McMaster University, Hamilton (CA)

(72) Inventors: Rabia Mateen, Brantford (CA); Monsur Ali, Hamilton (CA); Todd Hoare, Ancaster (CA)

(21) Appl. No.: 16/786,049

(22) Filed: Feb. 10, 2020

Related U.S. Application Data
(60) Provisional application No. 62/802,942, filed on Feb. 8, 2019.

(51) Int. Cl. G01N 33/544; C12Q 1/34; C08F 220/28; C08J 3/24; C08J 3/075

(52) U.S. Cl. CPC G01N 33/544; C12Q 1/34; C08F 220/282; C08J 2433/14; C08J 3/075; C08J 2333/14; C08J 3/246

(57) ABSTRACT

The invention pertains to a printable hydrogel that can both immobilize and stabilize a wide range of biomolecules and/or cells on a substrate while restricting the access of surrounding chemicals to the biomolecule active site. Such hydrogels can be adapted to high-throughput screening applications and can discriminate between true inhibitors and promiscuous aggregating inhibitors as well as enable the determination of dose-response relationships of biomolecule and/or cell inhibitory chemicals with high accuracy.

### CROSS-REFERENCE TO RELATED APPLICATION

The present application claims the benefit of U.S. provisional patent application No. 62/802,942, filed Feb. 8, 2019, the contents of which are herein incorporated by reference in its entirety.

### FIELD

This invention relates to printable hydrogel formulations that can optionally entrap biomolecules and/or cells to enable both bio-immobilization and maintenance of bioactivity.

### BACKGROUND

The effective immobilization of biomolecules such as proteins, polynucleotides, enzymes, etc. has significant implications in diverse fields including energy production, analytical assays, pharmaceutical synthesis, and drug screening.¹⁻⁴ In particular, enzyme immobilization within protein arrays⁵ has attracted interest in the biosensing field.

Physical entrapment of enzymes or other target biomolecules in a polymer network is particularly attractive due to the mild immobilization conditions required.⁶ Hydrogel-based enzyme immobilization platforms offer particular promise. The high water binding capacity of hydrogels can maintain biomolecule hydration over a broad range of storage/application conditions:⁷⁻⁹ promote high biomolecule mobility and flexibility,¹⁰ and maintain physiologically-mimetic conditions for optimal biomolecule activity for reaction catalysis¹¹ or target binding. In addition, the tunable porosity of hydrogels can enable selective transport of substrates to and from the biomolecule via size selectivity.¹² Interfacial thin film hydrogels are particularly attractive since they can minimize the kinetic/diffusional drawbacks associated with the use of bulk hydrogels in biosensing applications¹³ while maintaining the benefits of size selectivity.¹⁴ Several methods have been developed to fabricate thin-layer interfacial hydrogels on various substrates, including dip-coating,¹⁵ spray deposition,¹⁶ spin-coating¹⁷ and drop-on demand printing.¹⁸ Printing is particularly advantageous since it is amenable to dispensing small volumes (minimizing sample volumes for screening), can localize materials in specific patterns (enabling, for example, facile printing of multi-sample arrays on a substrate), and can be scaled to commercial production.¹⁹⁻²¹

There is interest in the drug discovery community to adopt protein arrays²² for high-throughput screening in place of the traditional slower and higher volume microplate assays.²³ A critical challenge in high-throughput screening for drug discovery is the large preponderance of false-positive hits.²⁴ Many compounds that behave non-specifically, or “promiscuously”, have been identified as artefactual leads in high-throughput drug screening,²⁵ resulting in the investment of time and money on chasing lead compounds that are not actually functional inhibitors. Promiscuous inhibition is typically linked to the tendency for such compounds to self-associate and form colloidal aggregates that sterically, rather than biologically inhibit binding to active sites.²⁶ Significant effort has been invested in examining the nature of these aggregates and determining methods to identify compounds demonstrating aggregative potential,²⁷⁻³⁰ with only limited success. Computational models have been designed to predict the presence of these compounds in pharmaceutical libraries, but have been shown also to generate both false positive and negative results³¹. Furthermore, the addition of a non-ionic detergent can disrupt some colloidal aggregates³² but cannot fully prevent aggregation and has been shown to interfere with other assay components.³³

Alternately, cell immobilization in hydrogels is of significant relevance to both drug screening (with similar applications and challenges as listed above) and “organ-on-a-chip”-type assemblies in which the cell responses to various chemical or biological stimuli are screened in a 3D-like environment that is a better approximation of a native tissue.^{33a} Minimizing the volume of the hydrogel in such applications is essential to reduce the diffusional path length of nutrients to (and waste from) cells, ensuring the maintenance of cell viability during a particular screening application.

### SUMMARY

In one embodiment, the present invention describes polymers that can react to form a hydrogel upon mixing, a printing technique capable of delivering these hydrogels to an interface or substrate, and a bioactive biomolecule and/or cell that can be physically entrapped inside the formed hydrogel. By entrapping the biomolecule and/or cell inside the printable hydrogel, one or more of the following attributes may be leveraged in an application: (1) the biomolecule/cell is immobilized at the interface without being washed away in an aqueous environment; (2) the biomolecule/cell is protected from the action of degrading enzymes or other degradation stimuli, primarily by (but not limited to) sterically blocking access to the entrapped biomolecule/cell based on the pore size of the entrapping hydrogel; (3) the biomolecule/cell is protected from chemical denaturation via a combination of steric blocking and chemical interactions between the biomolecule/cell and the gel phase; (4) the biomolecule/cell is protected from denaturation or membrane destabilization via drying, primarily by (but not limited to) the capacity of the entrapping hydrogel to maintain a hydrated environment around the biomolecule/cell; (5) the biomolecule/cell activity is not inhibited by physical aggregates or other species in the environment that can sterically, instead of chemically, block the active site of the biomolecule or receptors on cell surfaces.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows an ¹H-NMR spectra of poly(oligoethylene glycol methacrylate) polymers in one embodiment of the disclosure.

FIG. 2 shows a thin layer in situ gelling hydrogel printed on nitrocellulose substrate in one embodiment of the disclosure.

FIG. 3 shows the ATR-FTIR spectra of nitrocellulose paper substrate, POA, POH and POA+POH printed on nitrocellulose in one embodiment of the disclosure.

FIG. 4 shows the high-resolution XPS spectra of printed polymers.

FIG. 5 shows graphs indicating that printed hydrogels immobilize and stabilize molecules of varying sizes in one embodiment of the disclosure.

FIG. 6 shows the chromatography of polymers inks mixed with fluorescein (F) in one embodiment of the disclosure.

FIG. 7 shows the cross-sectional confocal microscopy images of printed hydrogel microzones.

FIG. 8 shows a graph indicating that printing β-lactamase in a hydrogel minimizes enzyme leaching in one embodiment of the disclosure.

FIG. 9 shows graphs indicating that the printed hydrogel protects enzyme (E) against proteolytic degradation and supports enzyme stabilization for long-term storage in one embodiment of the disclosure.

FIG. 10 shows images of maintained viable cells of multiple cell types inside the printed hydrogels over multiple days suitable for screening applications in one embodiment of the disclosure.

FIG. 11 shows a graph indicating that the printed hydrogel protects β-lactamase against chaotropic agent-induced denaturation.

FIG. 12 shows the printable hydrogel microarray for drug screening in one embodiment of the disclosure.

FIG. 13 shows graphs indicating that printed hydrogel-based β-lactamase screening assay can determine dose-response relationships of classic β-lactamase inhibitors and discriminate between true and promiscuous aggregating inhibitors in one embodiment of the disclosure.

FIG. 14 shows the selection of the optimal β-lactamase concentration in a printed hydrogel-based screening assay in one embodiment of the disclosure.

FIG. 15 shows a graph indicating the particle size distribution of promiscuous aggregating inhibitors (Rottlerin, BIS IX, TIPT).

### DETAILED DESCRIPTION

### (I) Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “alkyl” as used herein, whether it is used alone or as part of another group, means straight or branched chain, saturated alkyl groups, and includes for example, methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like. The term C₁₋₆alkyl means an alkyl group having 1, 2, 3, 4, 5, or 6 carbon atoms.

The term “alkylene” as used herein, whether alone or as part of another group, means an alkyl group that is bivalent; i.e. that is substituted on two ends with another group. The term Co₀₋₂alkylene means an alkylene group having 0, 1 or 2 carbon atoms. It is an embodiment of the application that, in the alkylene groups, one or more, including all, of the hydrogen atoms are optionally replaced with F or ²H.

The term “aryl” as used herein means a monocyclic, bicyclic or tricyclic aromatic ring system containing, depending on the number of atoms in the rings, for example from 6 to 10 carbon atoms, and at least 1 aromatic ring and includes, but is not limited to, phenyl, naphthyl, anthracenyl, 1,2-dihydronaphthyl, 1,2,3,4-tetrahydronaphthyl, fluorenyl, indanyl, indenyl and the like.

The term “heteroaryl” as used herein refers to cyclic groups that contain at least one aromatic ring and at least one heteroatom, such as N, O and/or S. The term C₅₋₁₀heteroaryl means an aryl group having 5, 6, 7, 8, 9 or 10 atoms, in which at least one atom is a heteroatom, such as N, O and/or S, and includes, but is not limited to, thienyl, furyl, pyrrolyl, pyrididyl, indolyl, quinolyl, isoquinolyl, tetrahydroquinolyl, benzofuryl, benzothienyl and the like.

The term “polymerizable” as used herein refers to the property of individual monomers to react with other monomers, whether the same or different, under appropriate conditions to yield polymers

The term “derivative” as used herein refers to a substance which comprises the same basic carbon skeleton and functionality as the parent compound, but can also bear one or more substituents or substitutions of the parent compound. For example, alkyl derivatives of oligoethylene glycol methacrylate would include any compounds in which an alkyl group is substituted on the oligoethylene glycol methacrylate backbone.

The term “precursor polymer” as used herein refers to an oligoethylene glycol methacrylate-based copolymer that has been modified to contain a reactive functional group, for example, a nucleophilic or electrophilic moiety. In one embodiment for example, a precursor polymer of the present disclosure comprises a hydrazide reactive group, or an aldehyde and/or ketone reactive functional group on a poly(oligoethylene glycol methacrylate) polymer.

The term “copolymer” as used herein is defined as a polymer derived from two or more different monomers. In one embodiment for example, a copolymer of the present disclosure includes a co-polymer of oligoethylene glycol methacrylate and acrylic acid. Other co-polymers include, for example, a co-polymer of oligoethylene glycol methacrylate and N-(2,2-dimethoxyethyl)methacrylamide (DMEMAm).

The term “nucleophile-functionalized” as used herein refers to a copolymer comprised of at least repeating units of oligoethylene glycol methacrylate in which a part of the copolymer has been functionalized with a nucleophilic moiety which can react with an electrophile or electrophilic moiety to form covalent cross-linked bonds.

The term “electrophile-functionalized” as used herein refers to a copolymer comprised of at least repeating units of oligoethylene glycol methacrylate in which a part of the copolymer has been functionalized with an electrophilic moiety which can react with a nucleophile or nucleophilic moiety to form covalent cross-linked bonds.

The term “polymeric backbone” as used herein refers to the main chain of a suitable polymer comprising a series of covalently bonded atoms that together create the continuous chain (straight or branched) of the polymeric molecule.

The term “crosslinked” or “crosslink” as used herein is defined as a bond that links a first precursor polymer to a second precursor polymer. The bonds can be covalent bonds. For example, the “crosslink” is a reversible hydrazone bond formed between a reactive hydrazide, and aldehyde and/or ketone functional groups.

The term “hydrogel” as used herein refers to a polymeric material that exhibits the ability to swell and retain a significant fraction of water within its structure, without dissolving in water.

The term “w/w” as used herein means the number of grams of solute in 100 g of solution.

The term “w/v” as used herein refers to the number of grams of solution in 100 mL of solvent.

The term “biomolecule” as used herein refers to an organic molecule that may be found in a living organism or synthetically produced and has biological activity.

### (II) Printed Hydrogels

The present disclosure is directed to hydrogels, and in particular, hydrogels that form a gel on a substrate and which are then able to immobilize a bioactive molecule.

Accordingly, in one embodiment, the present disclosure includes a hydrogel that:
a) forms a gel on a substrate from precursor polymer building block(s);
b) immobilizes a bioactive biomolecule and/or cell; and
c) controls access to the biomolecule and/or cell by other chemicals in the hydrogel environment.

The present disclosure also includes a drug screening platform, comprising:
a) a substrate;
b) a hydrogel printed on the substrate; and
c) a biomolecule and/or cell entrapped in the hydrogel.

In one embodiment, the hydrogel is an in situ gelling hydrogel.

In another embodiment, the hydrogel is printable.

In another embodiment, the hydrogel is protein-repellent.

In one embodiment, the hydrogel comprises poly(ethylene glycol), poly(oligoethylene glycol acrylate), poly(oligoethylene glycol methacrylate), poly(sulfobetaine), poly(carboxybetaine), or derivatives thereof

In another embodiment, the hydrogel is formed by mixing two covalently crosslinkable functionalized pre-polymers.

In another embodiment, the hydrogel comprises:
a. at least one first precursor polymer which is a hydrazide-functionalized poly(oligoethylene glycol methacrylate) copolymer, and
b. a second precursor polymer which is an aldehyde- and/or ketone-functionalized poly(oligoethylene glycol methacrylate) copolymer, wherein the first and second precursor polymers are crosslinked through hydrazone bonds to form the hydrogel.

In one embodiment, the first precursor polymer is a copolymer comprising monomeric units of:
a. a first monomer which is oligoethylene glycol methacrylate, or a derivative thereof and
b. at least one second polymerizable monomer which is functionalized, or is capable of being functionalized, with a nucleophilic moiety.

In an embodiment, the first monomer has the structure of the formula (I):

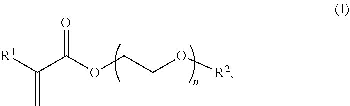

wherein
R¹ is H, (C₁-C₁₀)alkyl or (C₂-C₁₀)alkynyl;
R² is H, (C₁-C₁₀)alkyl, (C₂-C₁₀)alkynyl, -(C₀-C₄)-alkylene-(C₆-C₁₀)aryl, -(C₀-C₄)-alkylene-(C₅-C₁₀)heteroaryl, —C(O)NR′ or —C(O)OR′, wherein R′ is H or (C₁-C₆)alkyl, and
n is any integer between 6 and 30.

In another embodiment, R¹ is H, (C₁-C₆)alkyl or (C₂-C₆)alkynyl. In a further embodiment, R¹ is H or (C₁-C₄)alkyl. In another embodiment, R¹ is H or CH₃. In another embodiment, R¹ is CH₃. In one embodiment, R¹ is H.

In another embodiment, R² is H, (C₁-C₆)alkyl, (C₂-C₆)alkynyl, -(C₀-C₂)-alkylene-(C₆-C₁₀)aryl, —C(O)NR′ or —C(O)OR′, wherein R′ is H or (C₁-C₄)alkyl. In a further embodiment, R² is H, (C₁-C₄)alkyl, -(C₀-C₂)-alkylene-phenyl, —C(O)NR′ or —C(O)OR′, wherein R′ is H or (C₁-C₄)alkyl. In a further embodiment, R² is H or CH₃.

In one embodiment, n is any integer between 6 and 20, or between 6 and 12.

In another embodiment, the second polymerizable monomer is functionalized, or is capable of being functionalized, with a nucleophilic moiety, wherein the nucleophilic moiety is hydrazine or amine derivative, a carbonyl hydrate, an alcohol, cyanohydrin or cyanohydrin derivative, a thiol or thiol derivative, or a phosphorus ylide or derivatives thereof. In another embodiment, the nucleophilic moiety is a hydrazide.

In another embodiment, the first precursor polymer is a copolymer comprising monomeric units of:
a. a first monomer which is oligoethylene glycol methacrylate, or a derivative thereof; and
b. at least one second polymerizable monomer which is functionalized, or is capable of being functionalized, with a hydrazide moiety.

In one embodiment, the second polymerizable monomer has a carboxylic acid moiety, as the carboxylic acid can be functionalized to a hydrazide moiety. In another embodiment, the second polymerizable monomer is acrylic acid or a derivative thereof, methacrylic acid, itaconic acid, fumaric acid, maleic acid, or vinylacetic acid. In a further embodiment, the second monomer is acrylic acid or a derivative thereof. In another embodiment, the second polymerizable moiety is vinyl alcohol or allylic alcohol, which can be functionalized to a hydrazide moiety. In another embodiment, the second polymerizable moiety contains a nucleophilic moiety, such as a hydrazide moiety. In one embodiment, the second polymerizable moiety is acrylic acid functionalized with a hydrazide moiety

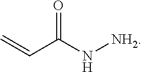

In another embodiment, the second polymerizable moiety of the first precursor polymer is

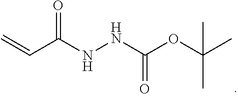

In another embodiment of the disclosure, the first precursor polymer is a co-polymer which further comprises a third monomer which has the structure of the formula (II):

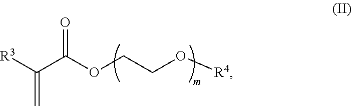

wherein
R³ is H, (C₁-C₁₀)alkyl or (C₂-C₁₀)alkynyl;
R⁴ is H, (C₁-C₁₀)alkyl, (C₂-C₁₀)alkynyl , -(C₀-C₄)-alkylene-(C₆-C₁₀)aryl, -(C₀-C₄)-alkylene-(C₅-C₁₀)heteroaryl, —C(O)NR′ or —C(O)O—R′, wherein R′ is H or (C₁-C₁-C₆)alkyl, and
m is any integer between 3 and 5.

In another embodiment, R³ is H, (C₁-C₆)alkyl or (C₂-C₆)alkynyl. In a further embodiment, R³ is H or (C₁-C₄)alkyl. In another embodiment, R³ is H or CH₃. In another embodiment, R³ is CH₃. In one embodiment, R³ is H.

In another embodiment, R⁴ is H, (C₁-C₆)alkyl, (C₂-C₁₆)alkynyl, -(C₀-C₄)-alkylene-(C₆-C₁₀)aryl, —C(O)NR′ or —C(O)O—R′, wherein R′ is H or (C₁-C₄)alkyl. In a further embodiment, R⁴ is H, (C₁-C₄)alkyl, -(C₀-C₄)-alkylene-phenyl, —C(O)NR′ or —C(O)O—R′, wherein R′ is H or (C₁-C₄)alkyl. In a further embodiment, and R⁴ is H or CH₃.

In another embodiment of the disclosure, the second precursor polymer is a copolymer comprising monomeric units of:
a. a first monomer which is oligoethylene glycol methacrylate, or a derivative thereof; and
b. a second polymerizable monomer which is functionalized, or is capable of being functionalized, with an electrophilic moiety.

In another embodiment, the second polymerizable monomer is functionalized, or is capable of being functionalized, with an electrophilic moiety, wherein the electrophilic moiety is an aldehyde, a ketones, a carboxylic acid, an ester, an amides, a maleimide, an acyl (acid) chloride, an acid anhydride, or an alkene or derivatives thereof. In another embodiment, the electrophilic moiety is an aldehyde or a ketone moiety.

In an embodiment, the second precursor polymer is a copolymer comprising monomeric units of:
a. a first monomer which is oligoethylene glycol methacrylate, or a derivative thereof; and
b. a second polymerizable monomer which is functionalized, or is capable of being functionalized, with an electrophilic moiety, in which the electrophilic moiety is an aldehyde or a ketone moiety.

In an embodiment, the first monomer has the structure of the formula (I):

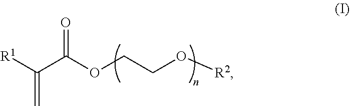

wherein
R¹ is H, (C₁-C₁₀)alkyl or (C₂-C₂₋₁₀)alkynyl;
R² is H, (C₁-C₁₀)alkyl, (C₂-C₁₀)alkynyl, -(C₀-C₄)-alkylene-(C₆-C₁₀)aryl, -(C₀-C₄)-alkylene-(C₅-C₁₀)heteroaryl, —C(O)NR′ or —C(O)OR′, wherein R′ is H or (C₁-C₆)alkyl, and
n is any integer between 6 and 30.

In another embodiment, R¹ is H, (C₁-C₆)alkyl or (C₂-C₆)alkynyl. In a further embodiment, R¹ is H or (C₁-C₄)alkyl. In another embodiment, R¹ is H or CH₃.

In another embodiment, R¹ is CH₃. In one embodiment, R¹ is H.

In another embodiment, R² is H, (C₁-C₆)alkyl, (C₂-C₆)alkynyl, -(C₀-C₂)-alkylene-(C₆-C₁₀)aryl, —C(O)NR′ or —C(O)OR′, wherein R′ is H or (C₁-C₄)alkyl. In a further embodiment, R² is H, (C₁-C₄)alkyl, -(C₀-C₂)-alkylene-phenyl, —C(O)NR′ or —C(O)OR′, wherein R′ is H or (C₁-C₄)alkyl. In a further embodiment, and R² is H or CH₃.

In one embodiment, n is any integer between 6 and 20, or between 6 and 12.

In an embodiment, the second polymerizable monomer is functionalized with an acetal moiety or a ketal moiety, as these moieties can be converted, after polymerization, to aldehyde or ketone moieties. In a further embodiment, the second polymerizable monomer is N-(2,2-dimethoxyethyl)methacrylamide (DMEMAm), allylic aldehyde or (N-((2-methyl-1,3-dioxolan-2-yl)methyl)methacrylamide).

In another embodiment of the disclosure, the second precursor polymer is a co-polymer which further comprises a third monomer which has the structure of the formula (II):

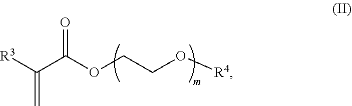

wherein
R³ is H, (C₁-C₁₀)alkyl or (C₂-C₁₀)alkynyl;
R⁴ is H, (C₁-C₁₀)alkyl, (C₂-C₁₀)alkynyl , -(C₀-C₄)-alkylene-(C₆-C₁₀)aryl, -(C₀-C₄)-alkylene-(C₅-C₁₀)heteroaryl, —C(O)NR′ or —C(O)O—R′, wherein R′ is H or (C₁-C₆)alkyl, and
m is any integer between 3 and 5.

In another embodiment, R³ is H, (C₁-C₆)alkyl or (C₂-C₆)alkynyl. In a further embodiment, R³ is H or (C₁-C₄)alkyl. In another embodiment, R³ is H or CH₃. In another embodiment, R³ is CH₃. In one embodiment, R³ is H.

In another embodiment, R⁴ is H, (C₁-C₆)alkyl, (C₂-C₁₆)alkynyl, -(C₀-C₄)-alkylene-(C₆-C₁₀)aryl, —C(O)NR′ or —C(O)O—R′, wherein R′ is H or (C₁-C₄)alkyl. In a further embodiment, R⁴ is H, (C₁-C₄)alkyl, -(C₀-C₄)-alkylene-phenyl, —C(O)NR′ or —C(O)O—R′, wherein R′ is H or (C₁-C₄)alkyl. In a further embodiment, and R⁴ is H or CH₃.

In one embodiment, the hydrogel is formed by sequential printing of the two covalently crosslinkable functionalized pre-polymers.

In another embodiment, the hydrogel is formed using in situ-gelling or click chemistry.

In another embodiment, the hydrogel is crosslinked by hydrazone bonds.

In one embodiment, the hydrogel is formed using sequential printing of aldehyde-functionalized poly(oligoethylene glycol methacrylate) and hydrazide-functionalized poly(oligoethylene glycol methacrylate).

In another embodiment, the hydrogel is printed by a solenoid drop-on-demand printer.

In another embodiment, the substrate comprises cellulose, nitrocellulose, cellulose acetate, glass, polysulfone, polyacrylonitrile, polystyrene, polypropylene, or polyethylene.

In another embodiment, the substrate is porous.

In another embodiment, the hydrogel is printed on the substrate in a microarray format. In one embodiment, the hydrogel printed microarray format can be incorporated into conventional high-throughput screening assays.

In another embodiment, the bioactive biomolecule is a protein, enzyme, DNA, RNA, aptamer, other polynucleotide, carbohydrate, proteoglycan, or glycoprotein.

In another embodiment, the bioactive component is a cell.

In another embodiment, the access to entrapped biomolecules can be sterically controlled by controlling the pore size of the hydrogel.

In another embodiment, the hydrogel can encapsulate stabilized biomolecules.

In another embodiment, the hydrogel can protect encapsulated biomolecules from proteolytic degradation.

In another embodiment, the hydrogel can protect encapsulated biomolecules from time-dependent denaturation.

In another embodiment, the hydrogel can protect encapsulated biomolecules from chaotropic agent denaturation.

In another embodiment, the stabilized biomolecules are enzymes.

In another embodiment, the hydrogel can encapsulate cells

In another embodiment, the hydrogel can protect cells from lysis and dehydration.

In another embodiment, the quantitative measurements of IC₅₀ values of real inhibitors of the encapsulated enzyme are enabled.

In another embodiment, true and promiscuous inhibitors of enzymes can be distinguished.

### (III) Method for Drug Candidate Screening

The present disclosure also includes a method for screening drug candidates against a bioactive biomolecule. In one embodiment, the method comprises
a) printing a hydrogel on a substrate, wherein the hydrogel is embedded with a biomolecule;
b) depositing a solution of a drug candidate and an analyte specific to the biomolecule and/or cell on the printed hydrogel;
c) quantitatively assessing the activity of the drug candidate on the biomolecule and/or cell.

In one embodiment, the analyte specific to the biomolecule and/or cell allows for the quantitative determination of the activity of the biomolecule and/or viability of the cell. In one embodiment, the analyte specific to the biomolecule and/or cell is a colorimetric analyte.

In another embodiment, the method allows for the distinction between a true inhibitor (or modifier) of the activity of the biomolecule and/or cell, and a promiscuous inhibitor or modifier of the biomolecule and/or cell. In another embodiment, the biomolecule is the enzyme β-lactamase and the method allows for the identification of true inhibitors of the enzyme.

In one aspect of the invention, the hydrogel is formed using in situ-gelling pairs of functionalized precursor polymers that can spontaneously crosslink upon co-delivery or sequential delivery to the interface to form a hydrogel. In one embodiment, the pore size (related to crosslink density) of the hydrogel can be systematically controlled in order to regulate what size of compounds or aggregates can and cannot access the entrapped biomolecule. In addition, the hydrogel chemistry is also chosen to exhibit protein-repellent properties to minimize the non-specific binding of proteins that may also sterically inhibit transport of a substrate, probe, or biomarker into or out of the hydrogel. In one embodiment, hydrazone crosslinked poly(oligoethylene glycol methacrylate) chemistry can contribute to each of these beneficial properties. In one embodiment, the hydrazide and aldehyde-functionalized poly(oligoethylene glycol methacrylate) (PO) polymers are used, as PO-based polymers exhibit high non-specific protein adsorption and hydrazide and aldehyde groups react rapidly upon mixing in water at ambient conditions to form hydrazone crosslinks (enabling printing).

In another embodiment, the hydrogel comprises:
a. at least one first precursor polymer which is a hydrazide-functionalized poly(oligoethylene glycol methacrylate) copolymer, and
b. a second precursor polymer which is an aldehyde- and/or ketone-functionalized poly(oligoethylene glycol methacrylate) copolymer,
wherein the first and second precursor polymers are crosslinked through hydrazone bonds to form the hydrogel.

In another embodiment, the pore size of the printed hydrogel is controlled by the amount of cross-linking of the hydrogel.

In another embodiment, the pore size of the printed hydrogel is controlled by the molecular weight of the hydrogel precursor polymers.

In one embodiment, the first precursor polymer is a copolymer comprising monomeric units of:
a. a first monomer which is oligoethylene glycol methacrylate, or a derivative thereof; and
b. at least one second polymerizable monomer which is functionalized, or is capable of being functionalized, with a nucleophilic moiety.

In an embodiment, the first monomer has the structure of the formula (I):

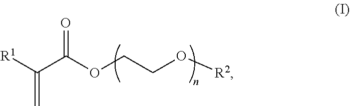

wherein
R¹ is H, (C₁-C₁₀)alkyl or (C₂-C₁₀)alkynyl;
R² is H, (C₁-C₁₀)alkyl, (C₂-C₁₀)alkynyl, -(C₀-C₄)-alkylene-(C₆-C₁₀)aryl, -(C₀-C₄)-alkylene-(C₅-C₁₀)heteroaryl, —C(O)NR′ or —C(O)OR′, wherein R′ is H or (C₁-C₆)alkyl, and
n is any integer between 6 and 30.

In another embodiment, R¹ is H, (C₁-C₆)alkyl or (C₂-C₆)alkynyl. In a further embodiment, R¹ is H or (C₁-C₄)alkyl. In another embodiment, R¹ is H or CH₃. In another embodiment, R¹ is CH₃. In one embodiment, R¹ is H.

In another embodiment, R² is H, (C₁-C₆)alkyl, (C₂-C₆)alkynyl, -(C₀-C₂)-alkylene-(C₆-C₁₀)aryl, —C(O)NR′ or —C(O)OR′, wherein R′ is H or (C₁-C₄)alkyl. In a further embodiment, R² is H, (C₁-C₄)alkyl, -(C₀-C₂)-alkylene-phenyl, —C(O)NR′ or —C(O)OR′, wherein R′ is H or (C₁-C₄)alkyl. In a further embodiment, R² is H or CH₃.

In one embodiment, n is any integer between 6 and 20, or between 6 and 12.

In another embodiment, the second polymerizable monomer is functionalized, or is capable of being functionalized, with a nucleophilic moiety, wherein the nucleophilic moiety is hydrazine or amine derivative, a carbonyl hydrate, an alcohol, cyanohydrin or cyanohydrin derivative, a thiol or thiol derivative, or a phosphorus ylide or derivatives thereof. In another embodiment, the nucleophilic moiety is a hydrazide.

In another embodiment, the first precursor polymer is a copolymer comprising monomeric units of:
a. a first monomer which is oligoethylene glycol methacrylate, or a derivative thereof and
b. at least one second polymerizable monomer which is functionalized, or is capable of being functionalized, with a hydrazide moiety.

In one embodiment, the second polymerizable monomer has a carboxylic acid moiety, as the carboxylic acid can be functionalized to a hydrazide moiety. In another embodiment, the second polymerizable monomer is acrylic acid or a derivative thereof, methacrylic acid, itaconic acid, fumaric acid, maleic acid, or vinylacetic acid. In a further embodiment, the second monomer is acrylic acid or a derivative thereof. In another embodiment, the second polymerizable moiety is vinyl alcohol or allylic alcohol, which can be functionalized to a hydrazide moiety. In another embodiment, the second polymerizable moiety contains a nucleophilic moiety, such as a hydrazide moiety. In one embodiment, the second polymerizable moiety is acrylic acid functionalized with a hydrazide moiety.

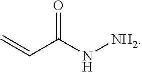

In another embodiment, the second polymerizable moiety of the first precursor polymer is

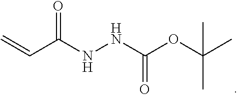

In another embodiment of the disclosure, the first precursor polymer is a co-polymer which further comprises a third monomer which has the structure of the formula (II):

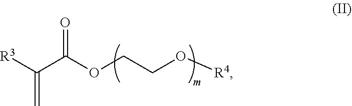

wherein
R³ is H, (C₁-C₁₀)alkyl or (C₂-C₁₀)alkynyl;
R⁴ is H, (C₁-C₁₀)alkyl, (C₂-C₁₀)alkynyl , -(C₀-C₄)-alkylene-(C₆-C₁₀)aryl, -(C₀-C₄)-alkylene-(C₅-C₁₀)heteroaryl, —C(O)NR′ or —C(O)O—R′, wherein R′ is H or (C₁-C₆)alkyl, and
m is any integer between 3 and 5.

In another embodiment, R³ is H, (C₁-C₆)alkyl or (C₂-C₆)alkynyl. In a further embodiment, R³ is H or (C₁-C₄)alkyl. In another embodiment, R³ is H or CH₃. In another embodiment, R³ is CH₃. In one embodiment, R³ is H.

In another embodiment, R⁴ is H, (C₁-C₆)alkyl, (C₂-C₆)alkynyl, -(C₀-C₄)-alkylene-(C₆-C₁₀)aryl, —C(O)NR′ or —C(O)O—R′, wherein R′ is H or (C₁-C₄)alkyl. In a further embodiment, R⁴ is H, (C₁-C₄)alkyl, -(C₀-C₄)-alkylene-phenyl, —C(O)NR′ or —C(O)—R′, wherein R′ is H or (C₁-C₄)alkyl. In a further embodiment, and R⁴ is H or CH₃.

In another embodiment of the disclosure, the second precursor polymer is a copolymer comprising monomeric units of:
a. a first monomer which is oligoethylene glycol methacrylate, or a derivative thereof; and
b. a second polymerizable monomer which is functionalized, or is capable of being functionalized, with an electrophilic moiety.

In another embodiment, the second polymerizable monomer is functionalized, or is capable of being functionalized, with an electrophilic moiety, wherein the electrophilic moiety is an aldehyde, a ketones, a carboxylic acid, an ester, an amides, a maleimide, an acyl (acid) chloride, an acid anhydride, or an alkene or derivatives thereof. In another embodiment, the electrophilic moiety is an aldehyde or a ketone moiety.

In an embodiment, the second precursor polymer is a copolymer comprising monomeric units of:
a. a first monomer which is oligoethylene glycol methacrylate, or a derivative thereof and
b. a second polymerizable monomer which is functionalized, or is capable of being functionalized, with an electrophilic moiety, in which the electrophilic moiety is an aldehyde or a ketone moiety.

In an embodiment, the first monomer has the structure of the formula (I):

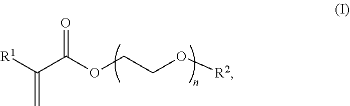

wherein
R¹ is H, (C₁-C₁₀)alkyl or (C₂-C₁₀)alkynyl;
R² is H, (C₁-C₁₀)alkyl, (C₂-C₁₀)alkynyl, -(C₀-C₄)-alkylene-(C₆-C₁₀)aryl, -(C₀-C₄)-alkylene-(C₅-C₁₀)heteroaryl, —C(O)NR′ or —C(O)OR′, wherein R′ is H or (C₁-C₆)alkyl, and
n is any integer between 6 and 30.

In another embodiment, R¹ is H, (C₁-C₆)alkyl or (C₂-C₆)alkynyl. In a further embodiment, R¹ is H or (C₁-C₄)alkyl. In another embodiment, R¹ is H or CH₃. In another embodiment, R¹ is CH₃. In one embodiment, R¹ is H.

In another embodiment, R² is H, (C₁-C₆)alkyl, (C₂-C₆)alkynyl, -(C₀-C₂)-alkylene-(C₆-C₁₀)aryl, —C(O)NR′ or —C(O)OR′, wherein R′ is H or (C₁-C₄)alkyl. In a further embodiment, R² is H, (C₁-C₄)alkyl, -(C₀-C₂)-alkylene-phenyl, —C(O)NR′ or —C(O)OR′, wherein R′ is H or (C₁-C₄)alkyl. In a further embodiment, and R² is H or CH₃.

In one embodiment, n is any integer between 6 and 20, or between 6 and 12.

In an embodiment, the second polymerizable monomer is functionalized with an acetal moiety or a ketal moiety, as these moieties can be converted, after polymerization, to aldehyde or ketone moieties. In a further embodiment, the second polymerizable monomer is N-(2,2-dimethoxyethyl)methacrylamide (DMEMAm), allylic aldehyde or (N-((2-methyl-1,3-dioxolan-2-yl)methyl)methacrylamide).

In another embodiment of the disclosure, the second precursor polymer is a co-polymer which further comprises a third monomer which has the structure of the formula (II):

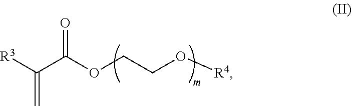

wherein
R³ is H, (C₁-C₁₀)alkyl or (C₂-C₁₀)alkynyl;
R⁴ is H, (C₁-C₁₀)alkyl, (C₂-C₁₀)alkynyl , -(C₀-C₄)-alkylene-(C₆-C₁₀)aryl, -(C₀-C₄)-alkylene-(C₅-C₁₀)heteroaryl, —C(O)NR′ or —C(O)O—R′, wherein R′ is H or (C₁-C₆)alkyl, and
m is any integer between 3 and 5.

In another embodiment, R³ is H, (C₁-C₆)alkyl or (C₂-C₆)alkynyl. In a further embodiment, R³ is H or (C₁-C₄)alkyl. In another embodiment, R³ is H or CH₃. In another embodiment, R³ is CH₃. In one embodiment, R³ is H.

In another embodiment, R⁴ is H, (C₁-C₆)alkyl, (C₂-C₆)alkynyl, -(C₀-C₄)-alkylene-(C₆-C₁₀)aryl, —C(O)NR′ or —C(O)O—R′, wherein R′ is H or (C₁-C₄)alkyl. In a further embodiment, R⁴ is H, (C₁-C₄)alkyl, -(C₀-C₄)-alkylene-phenyl, —C(O)NR′ or —C(O)O—R′, wherein R′ is H or (C₁-C₄)alkyl. In a further embodiment, and R⁴ is H or CH₃.

In another aspect of the invention, any printing method compatible with the gelation chemistry used may be applied, including (but not limited to) dip-coating, dip pen or contact lithographic techniques, spray deposition, spin-coating, thermal or piezoelectric inkjet printing, flexographic printing, or drop-on demand printing.¹⁸ In one embodiment, drop-on-demand solenoid printing is used given its capacity to rapidly deliver controlled volumes of gel precursor polymers while avoiding some of the issues associated with other printing techniques (e.g. lack of localization capacity, heating upon printing that may destabilize biomolecules, etc.). Sequential printing of the two reactive pre-polymers is used in one embodiment, although co-delivery with appropriate nozzle design would be similarly effective. Any substrate may be used for the printing method; nitrocellulose is used in one embodiment, although any substrate that can effectively anchor to the first printed layer (cellulose-based, polymer-based, glass, or silicone-based) would have similar efficacy.

In a further aspect of the invention, the biomolecule is selected from proteins, antibodies, enzymes, DNA, RNA, aptamers, other polynucleotides, carbohydrates, glycoproteins, proteoglycans, or any other biomolecule with some kind of bioactivity (i.e. enzymatic, binding affinity, transport, etc.) useful in a specific application such as, but not limited to, catalysis, biosensing, bioactivity screening, or fundamental studies of biomolecular interactions. In one embodiment, the biomolecule is physically mixed with one or more of the precursor polymers and/or sequentially printed between the reactive polymer precursors that can form the hydrogel to enable physical immobilization within the gel network. Chemical interactions with the biomolecule may optionally be promoted based on the choice of polymer and crosslinking chemistry and may be useful for stabilizing the biomolecule structure and/or enhancing biomolecule retention inside the gel; however, such chemical interactions are not a required attribute of the invention. In one embodiment, the biomolecule is an enzyme, for example β-lactamase.

In a further aspect of the invention, one or more types of cells may be physically mixed with one or more of the precursor polymers and/or sequentially printed between the reactive polymer precursors that can form the hydrogel to enable physical immobilization within the gel network.

In a further embodiment, a microarray of hydrogel-entrapped biomolecules and/or cells, which may be duplicates of the same gel/biomolecule and/or cell composition or a variety of different gel/biomolecule and/or cell compositions, is printed and used for biological screening applications. In another embodiment, the hydrogels are printed inside templates of conventional 96-well or 384-well multi-well plates fabricated on the substrate by wax printing or any other hydrophobic barrier technique. In this embodiment, the resulting biomolecule microarrays can be incorporated into current high-throughput screening geometries and protocols as desired.

In another embodiment, the printed hydrogel is used to establish a drug screening platform based on the enzyme, β-lactamase. This embodiment allows for quantitative measurement of the dose-response relationships of β-lactamase inhibitors with the same accuracy as higher volume solution assays. In addition, the printed enzyme immobilizing/stabilizing hydrogels can unambiguously identify non-specific inhibitors of β-lactamase that frequently appear as false-positive hits in many drug screening efforts, avoiding the current additional studies on these false hits that are both costly and time-consuming. More specifically, the printed hydrogel is able to discriminate between true inhibitors and a class of compounds called promiscuous aggregating inhibitors. These compounds form colloidal aggregates (typically but not exclusively ranging in size between 50-500 nm in aqueous solutions) and are responsible for non-mechanistic based enzymatic inhibition.

### EXAMPLES

The following non-limiting examples are illustrative of the present application:

### Example 1: Synthesis of poly(oligoethylene glycol methacrylate) Polymers

Unfunctionalized poly(oligoethylene glycol methacrylate) (PO) was prepared by adding azobis(methyl isobutyrate) (AIBMe) (50 mg, 0.22 mmol), oligo(ethylene glycol) methyl ether methacrylate OEGMA₄₇₅) (0.90 g, 1.9 mmol), di(ethylene glycol) methyl ether methacrylate (M(EO)₂MA) (3.1 g, 16.5 mmol) and thioglycolic acid (TGA) (7.5 μL, 0.15 mmol) to a 50 mL Schlenk flask. 1,4-Dioxane (20 mL) was added, and the solution was purged with nitrogen for 30 minutes. The flask was sealed and submerged in a pre-heated oil bath at 75° C. for 4 hours under magnetic stirring. After polymerization, the solvent was removed by rotary evaporation, and the poly(OEGMA₄₇₅-co-M(EO)₂MA) polymer was purified by dialysis against deionized water (DIW) for 6 cycles (6 hours/cycle) and lyophilized to dryness. The polymer was dissolved in 10 mM PBS at 20 w/w % and stored at 4° C.

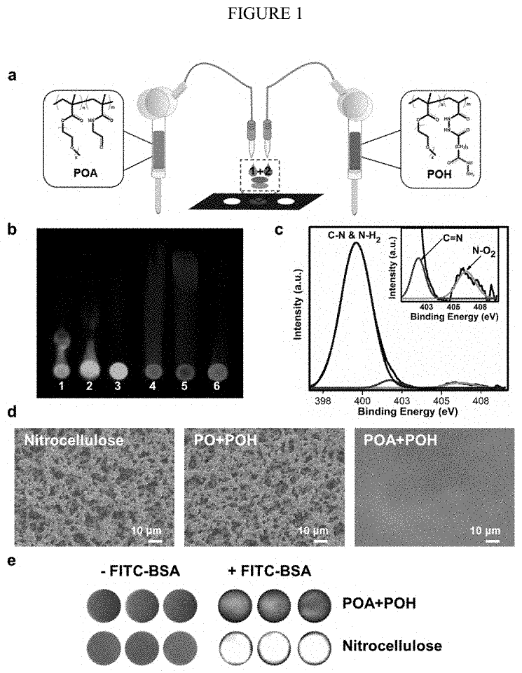

Aldehyde-functionalized poly(oligoethylene glycol methacrylate) (POA) was prepared similarly to the unfunctionalized PO polymer above except for the addition of N-(2,2- dimethoxyethyl)methacrylamide (DMEMAm) (0.63 g, 3.61 mmol). Following solvent removal, the acetal groups of the DMEMAm residues were converted to aldehydes via hydrolysis by dissolving the copolymer in 75 mL DIW and 25 mL 1.0 M HCl and stirring for 24 hours. The polymer was purified by dialysis against DIW and lyophilized to dryness. POA was dissolved in 10 mM PBS at 20 w/w % and stored at 4° C. The number-average molecular weight was determined to be 14 kDa (Ð)=2.03) from size exclusion chromatography. The aldehyde content was determined to be 12 mol % using ¹H-NMR, calculated by comparing the integration of the proton signals of the methoxy (O—CH₃, 3H, δ=3.3 ppm) and aldehyde (CHO, 1H, δ=9.2 ppm) groups (FIG. 1). FIG. 1 shows the ¹H-NMR spectra of poly(oligoethylene glycol methacrylate) polymers. (a) Unfunctionalized poly(oligoethylene glycol methacrylate) (PO); (b) Aldehyde-functionalized poly(oligoethylene glycol methacrylate) (POA); (c)

Hydrazide-functionalized poly(oligoethylene glycol methacrylate) (POH). Chemical shifts are reported relative to residual deuterated solvent peaks. Peak assignments are given on each spectrum based on the anticipated chemical structure of each polymer.

Hydrazide-functionalized poly(oligoethylene glycol methacrylate) (POH) was prepared by adding AIBMe (37 mg, 0.16 mmol), OEGMA₄₇₅ (0.90 g, 1.9 mmol), M(EO)₂MA (3.1 g, 16.5 mmol), acrylic acid (AA) (0.55 g, 7.6mmol), and TGA (7.5 μL, 0.15 mmol) to a 50 mL Schlenk flask. Polymerization proceeded similarly to that of PO and POA. Following solvent removal, the copolymer was dissolved in 100 mL DIW. Adipic acid dihydrazide (ADH) (4.33g, 24.8 mmol, 8.16 mol eq.) was added, and the pH of the solution was adjusted to 4.75. The reaction was initiated by the addition of 1-ethyl-3-(3-dimethylaminopropyl)carbodiimide (EDC) (1.93 g, 12.4 mmol, 3.80 mol eq.), after which the pH was maintained at 4.75 by the dropwise addition of 0.1 M HCl over 4 hours. The solution was left to stir overnight, dialyzed against DIW over 6 cycles (6 hours/cycle) and lyophilized to dryness. The polymer was dissolved in 10 mM PBS at 20 w/w % and stored at 4° C. The number-average molecular weight was determined to be 17 kDa (Ð=2.08) from size exclusion chromatography. The degree of hydrazide functionalization was determined to be 22 mol % by conductometric base-into-acid titration, comparing the carboxylic acid content before and after ADH conjugation (0.1 M NaOH titrant, 50 mg polymer in 50 mg of 1 mM NaCl titration solution, ManTech automatic titrator).

### Example 2: Hydrogel Printing

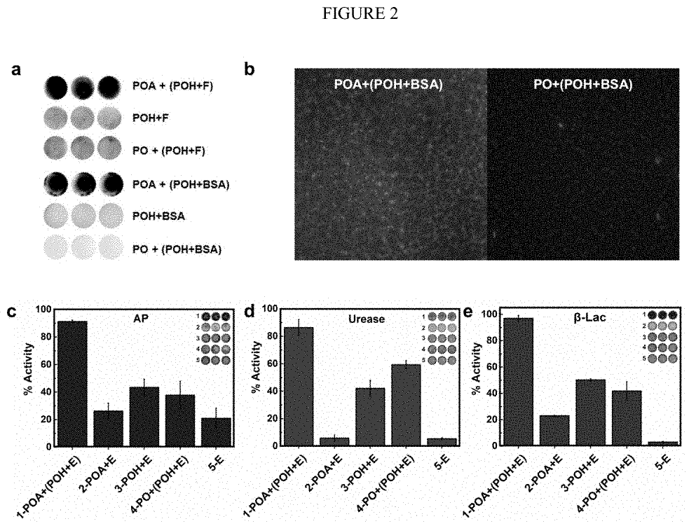

A paper microzone plate was first fabricated by printing hydrophobic wax barriers onto nitrocellulose membrane (EMD Millipore) using a Xerox ColorQube 8570N solid wax printer and a 96 well-plate template (3 mm diameter wells with ˜9 mm inter-well distance). The wax-printed paper was placed into an oven at 120 ° C. for 2 min to melt the wax through the paper. Polymer inks were composed of 6 w/w % aldehyde-functionalized poly(oligoethylene glycol methacrylate) (POA) or hydrazide-functionalized poly(oligoethylene glycol methacrylate) (POH), with 5 w/w % glycerol added as a humectant and viscosity modifier in both cases; the resulting viscosities of the POA and POH inks were 3.27 mPa·s and 4.85 mPa·s respectively (Vibro Viscometer SV-10/SV-100, A&D Company, Limited). A BioJet HR™ non-contact solenoid dispenser was used to print the inks onto the paper microzones (FIG. 2a). The two reagent lines were charged with POA and POH inks. The dispenser valve was programmed to stay open for 6 ms, and the frequency was set to 100 Hz. The thin-layer hydrogel was fabricated by dispensing 2 μL of POA onto the microzone, immediately followed by 2 μL of POH. The samples were dried and stored at room temperature. FIG. 2 shows a thin layer in situ gelling hydrogel printed on nitrocellulose substrate in one embodiment of the disclosure: (a) Schematic of aldehyde-functionalized poly(oligoethylene glycol methacrylate) (POA) and hydrazide-functionalized poly(oligoethylene glycol methacrylate) (POH) polymers sequentially printed onto a nitrocellulose paper substrate using a solenoid-controlled drop-on-demand printing system; (b) Chromatography of printed polymers in 70:30 methanol:water: 1. FITC-POH; 2. Unfunctionalized poly(oligoethylene glycol methacrylate) (PO)+FITC-POH; 3. POA+FITC-POH; 4. Rhodamine-POA; 5. PO+Rhodamine-POA; 6. POH+Rhodamine-POA; (c) High-resolution XPS spectra of P0A+POH collected in the N is region. The peak at 401.7 eV corresponds to the —C═N group characteristic of a hydrazone bond; (d) SEM images of printed samples after washing in 10 mM PBS; (e) Fluorescence scans of bare nitrocellulose and printed hydrogel (POA+POH) samples before and after incubation in 100 μg/mL FITC-BSA showing a significant reduction in non-specific protein adsorption upon hydrogel coating.

### Example 3: Printed Polymer Chromatography

Rhodamine-POA or FITC-POH were printed alone, with PO (unfunctionalized poly(oligoethylene glycol methacrylate)) or with the corresponding, unlabelled reactive polymer. Paper samples were cut into 0.5×4.5 cm strips, and chromatography was subsequently performed by placing the end of each strip in 50 μL of a 70:30 methanol:water solvent mixture. The samples were imaged through the fluorescein and rhodamine channels of the ChemiDoc™ MP System (BioRad). Image processing was performed in Image Lab™ software (Version 5.2, BioRad).

Gelation was validated by examining whether the fluorescently labeled polymers remained immobilized at their printed positions when exposed to the methanol-water chromatographic separation process. Fluorescently labeled POH (FITC-POH) or POA (Rhodamine-POA) polymers were printed alone, with an unfunctionalized PO polymer (incapable of covalent crosslinking with POH or POA), or with the corresponding unlabeled reactive polymer precursors (POH or POA) (FIG. 2b). When FITC-POH or Rhodamine-POA was printed alone (FIG. 2b, Columns 1 and 4) or with unfunctionalized PO polymer (FIG. 2b, Columns 2 and 5), the fluorescent precursor could transport up the nitrocellulose strip, indicating poor immobilization; conversely, when the reactive POA and POH polymers were sequentially printed (FIG. 2b, Columns 3 and 6), the labeled polymer remained localized at the printed site, suggesting effective gelation.

### Example 4: Characterization of Printed Hydrogels

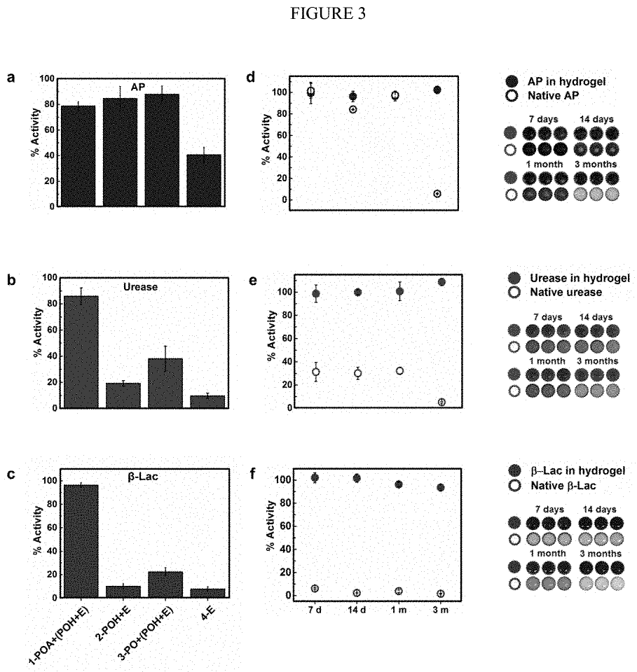

ATR-FTIR was performed on printed polymer samples following extensive washing with 10 mM PBS using a Vertex 70 FTIR Diamond ATR (Bruker) (FIG. 3). Each sample was subjected to 64 scans, and data were recorded with a 4 cm⁻¹ spectral resolution, with the decrease in intensity of both the nitro group and the cellulose —CH stretch peak relative to the carbonyl peak in the printed polymers confirming successful deposition of polymer at the surface. FIG. 3 shows the ATR-FTIR spectra of nitrocellulose paper substrate, POA, POH and POA+POH printed on nitrocellulose. In the nitrocellulose spectrum, the peak at 1340 cm⁻¹ corresponds to the nitro group (—NO₂) stretch while the peak at 2960 cm¹ corresponds to the —CH group stretch in the cellulose backbone. In the printed poly(oligoethylene glycol methacrylate) spectra, the peak at 1715 cm⁻¹ corresponds to the ester group (—C═O) stretch from the PO polymer side chain. Hydrazide and aldehyde groups also both appear in the range of the ester signals and are convoluted with these ester peaks; however, the C═O signal is primarily associated with the PO polymers rather than the nitrocellulose. The decrease in intensity of both the nitro group and the cellulose —CH stretch peak relative to the carbonyl peak in the printed polymers suggests that the polymers were successfully printed onto the nitrocellulose paper surface.

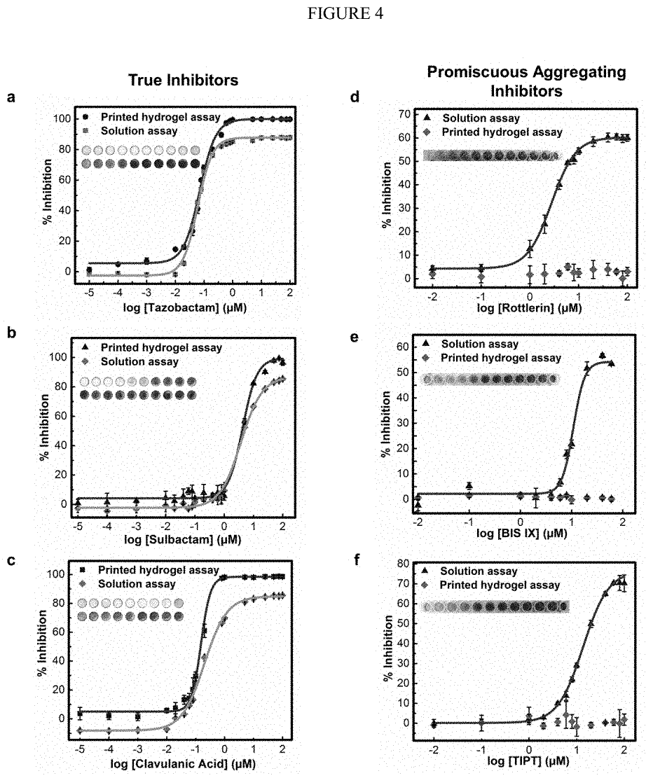

XPS spectra were recorded with a Physical Electronics (PHI) Quantera II spectrometer using a monochromatic Al K-α X-ray (1486.7 eV) source at 50 W (15 kV) (FIG. 2c, FIG. 4). The spectrometer was calibrated by assuming the binding energy of the Ag3d5/2 peak was at 368.0±0.1 eV and the full width at half maximum was at least 0.52 eV. Survey (280 eV pass energy), high-resolution carbon (26 eV pass energy) and high-resolution nitrogen (55 eV pass energy) XPS scans were obtained using a 45° take-off angle. Data analysis was performed using PHI MultiPak software (Version 9.4.0.7). Peak assignments were made according to the values reported in the NIST XPS Database. Samples sequentially printed with POA and POH indicated a peak in the high-resolution nitrogen spectrum at 401.7 eV that corresponds to the —C═N functional group characteristic of a hydrazone bond (FIG. 2c). FIG. 4 shows the high-resolution XPS spectra of printed polymers. (a) Survey scan of POA+POH; (b) Spectrum of POA+POH printed hydrogel samples collected in the C 1 s region. The peak at 286.1 eV corresponds to the —C═N group found in the hydrazone bond; (c) Spectrum of POA collected in the C 1 s region; (d) Spectrum of POA collected in N 1 s region. (e) Spectrum of POH collected in C 1 s region; (f) Spectrum of POH collected in N 1 s region; (g) Spectrum of nitrocellulose collected in C 1 s region; (h) Spectrum of nitrocellulose collected in N 1 s region.

The surface morphology of both printed and non-printed surfaces was evaluated by SEM (FEI-Magellan XHR SEM), using secondary electron image (SEI) mode with voltages of 2.0 kV (1000×magnification). SEM images of vigorously washed gel-printed nitrocellulose strips indicate that the rough and bulbous morphology of unmodified nitrocellulose remains unchanged when (unreactive) PO and POH are sequentially printed, consistent with these polymers being removed from the substrate during the washing step (FIG. 2d, panels 1 and 2); conversely, printing the (reactive) POA+POH pair results in significant smoothing of the substrate consistent with the formation of an interfacial gel layer (FIG. 2d, panel 3).

### Example 5: Protein Adsorption

The capacity of the printed hydrogels to resist non-specific protein adsorption was tested by fluorescently labeling a model protein and performing fluorescence imaging. Printed samples were soaked in 10 mM PBS for 12 hours, after which the hydrated samples were submerged in a 100 μg/mL solution of FITC-BSA and gently shaken for 2 hours. The samples were imaged through the fluorescein channel of the ChemiDoc™ MP System (BioRad). Image processing was performed using Image Lab™ software (Version 5.2, BioRad).

The printed hydrogel significantly suppresses non-specific protein adsorption to the nitrocellulose substrate (FIG. 2e), a notable benefit for optimizing the sensitivity of any bioassay by avoiding steric blocking of potential binding/diffusion sites for the target molecule. The printing method used here is both significantly faster (seconds as opposed to hours) and uniquely enables highly the localized gel printing essential for creating microarrays relative to previously reported methods for creating protein-repellent interfaces with similar chemistry.

### Example 6: Biomolecule Immobilization and Stabilization

### Fluorescein and FITC-BSA Entrapment Studies

POH ink solutions were prepared with a final concentration of 10 μM fluorescein or 0.05 mg/mL FITC-BSA. Samples printed with fluorescein were washed in 0.1 M NaOH+0.1% Tween 20, while samples printed with FITC-BSA were washed in 10 mM PBS and shaken at 300 rpm on an IKA MS3 Basic Shaker for 30 min.; each rinse solution was selected to maximize the solubility of the fluorescently-labeled probe and thus maximize the potential for washing the probe away from the surface if it was not effectively immobilized. Afterwards, both samples were imaged through the fluorescein channel of the ChemiDoc™MP System (BioRad). Image processing was performed in Image Lab™ software (Version 5.2, BioRad). FITC-BSA printed samples were also imaged with a Nikon Eclipse LV100ND optical microscope equipped with an Andor Zyla sCMOS camera at 20× magnification through the fluorescein channel to assess the distribution of FITC-BSA on the printed hydrogel surface. The 3D distribution of Rhodamine-POA and FITC-BSA within the printed gel layer was assessed using confocal fluorescence microscopy (CLSM, Nikon). Confocal z-stack images (3D view) were collected by scanning the printed gel samples at 10 μm intervals to a depth of 80 μm (326×326 μm area probed).

Chromatographic experiments confirming immobilization of encapsulated fluorophores upon gel printing were additionally performed by printing the relevant POA or POH solutions on a nitrocellulose paper substrate as described above, cutting the printed paper into 0.5×4.5 cm strips, and performing chromatography by dipping the end of the strip in 50 μL of a 50:50 methanol:water solvent. The samples were imaged through the fluorescein channel of the ChemiDoc™ MP System (BioRad). Image processing was performed using Image Lab™ software (Version 5.2, BioRad).

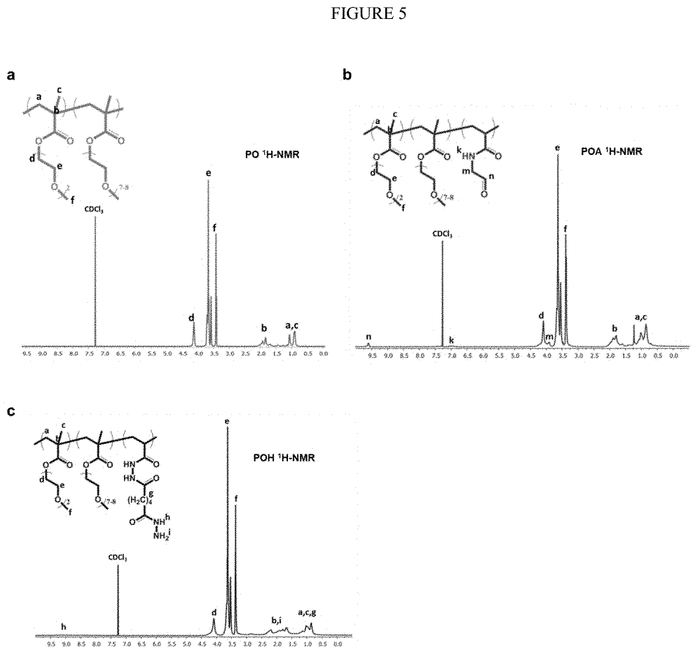

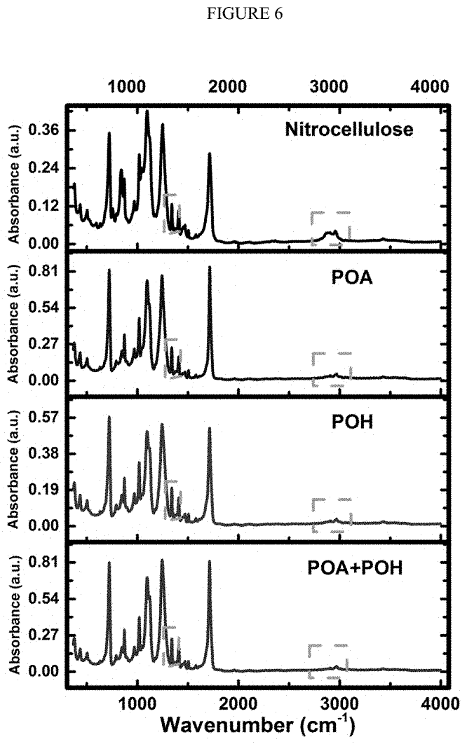

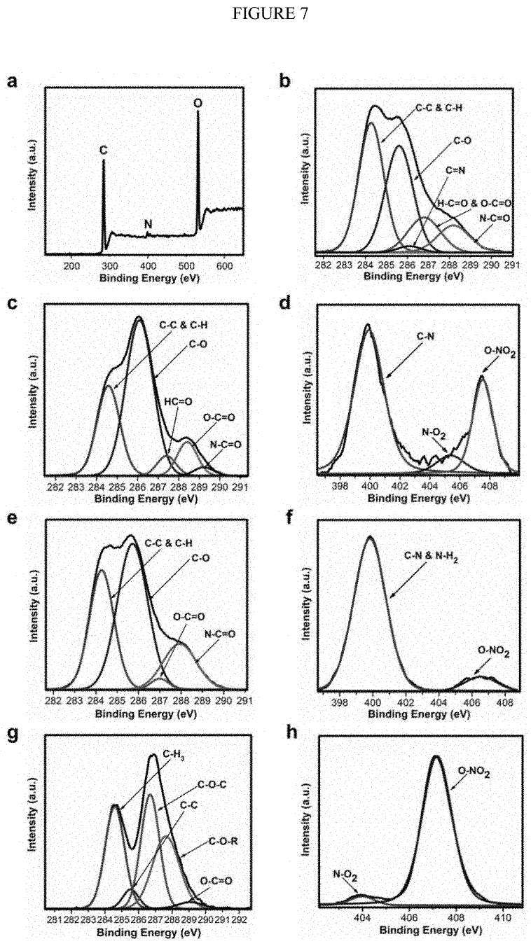

Both fluorescein (POA+(POH+F)) and BSA (POA+(POH+BSA)) remained entrapped in the crosslinked polymer assembly after the samples were washed vigorously, while the POH+F or POH+BSA ink printed alone or with an unreactive (PO) polymer could be almost entirely washed from the surface (FIG. 5a). Printed samples subjected to chromatographic separation similarly showed minimal transport of the fluorescent dopants from the gel-printed samples but rapid transport when the dopants were printed alone or with an unreactive PO polymer (FIG. 6). Fluorescence microscopy images of printed FITC-BSA confirmed the uniform distribution of the protein on the nitrocellulose surface when entrapped in the thin layer hydrogel (FIG. 5b), while confocal microscopy images of FITC-BSA encapsulated inside a hydrogel prepared with Rhodamine-POA confirm that the printed protein is distributed evenly throughout both the cross-section and the depth of the printed hydrogel microzones (FIG. 7). FIG. 5 shows graphs indicating that printed hydrogels immobilize and stabilize molecules of varying sizes in one embodiment of the disclosure: (a) Printed fluorescein (˜332 Da) after washing samples in 0.1 M NaOH+0.1% Tween 20 and printed FITC-BSA (˜66 kDa) after washing samples in 10 mM PBS for 10 min.(b) FITC-BSA printed in a gelling ink (left) and a non-gelling ink (right) imaged by a fluorescence microscope following washing (20× magnification); (c-e) Residual activity of enzymes (E) following washing of samples in 10 mM PBS for 10 min relative to the corresponding unwashed control: (c) Alkaline phosphatase (AP; ˜69 kDa); (d) Urease (˜546 kDa); (e) β-lactamase ((3-Lac; ˜29 kDa). Error bars represent the standard deviation from the mean (n=3). FIG. 6 shows the chromatography of polymers inks mixed with fluorescein (F) in one embodiment of the disclosure. In the pipetting experiment, polymer inks were mixed with fluorescein and the corresponding reactive or unreactive polymer and directly pipetted onto a nitrocellulose paper substrate. In the printing experiment, polymer inks were mixed with fluorescein and printed onto a nitrocellulose substrate, followed immediately by printing of the corresponding reactive or unreactive polymer. Chromatography was performed in a 50:50 methanol:water mixture. Lane 6 shows that the pipetted or printed polymer assembly (POA+POH) successfully immobilizes a large fraction of the fluorescein, while all other samples tested result in highly effective transport of essentially all of the printed fluorescein up the strip. FIG. 7 shows the cross-sectional confocal microscopy images of printed hydrogel microzones. FITC-BSA channel, Rhodamine-POA channel and overlaid fluorescence images confirm the co-localization of FITC-BSA within the gel as well as the relatively uniform distribution of FITC-BSA within the printed gel. Top (a) and bottom (b) views of the 326×326 μm cross-sectional slice imaged at a depth of 80 Mm.

### Example 7: Enzyme Entrapment Studies

POH ink solutions containing one of the tested model enzymes were prepared and printed as previously described, followed by washing with 10 mM PBS at 300 rpm on an IKA MS3 Basic Shaker for 10 minutes. The relevant substrate solutions for each enzyme were then pipetted onto the washed samples to assess enzyme activity. Images of the resulting colorimetric read-out were taken with an IPhone 5C camera. Image analysis to determine colorimetric intensity was performed using Fiji, an open-source program based on ImageJ. The converted substrate colour was extracted using the Color Deconvolution plugin. Extracted images were inverted and converted to 8 bit grayscale images. The intensity of each sample was measured and presented as a ratio of the corresponding control image (a sample printed in the same way but not washed to remove any non-encapsulated enzyme). In addition, printed samples were washed in 10 mM PBS for varying amounts of time, after which β-lactamase activity was assessed in the wash solutions via UV-vis spectrophotometry by tracking the hydrolysis of nitrocefin by monitoring solution absorbance at 492 nm. The resulting absorbance readings are reported as a ratio of the control (i.e. the absorbance of buffer itself at 492 nm).

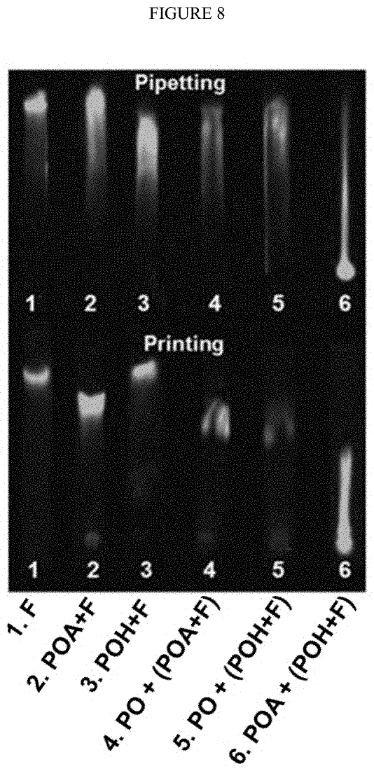

All tested enzymes were effectively immobilized and stabilized in the printed hydrogel (POA+(POH+E)), with >90% activity maintained for alkaline phosphatase (AP) and β-lactamase (β-Lac) and >85% activity maintained for urease relative to enzymes printed in the same manner but not rinsed prior to activity testing (FIG. 5c-e). Note that although nitrocellulose has a high capacity for protein retention, printed enzyme did not remain associated with unmodified nitrocellulose after washing; as such, the observation of residual enzyme activity after washing confirms effective enzyme entrapment. High entrapment efficiencies were also confirmed via washing experiments in which enzyme activity was assayed in sequential wash solutions; minimal activity losses are observed after the first 10 minute wash cycle (which removes poorly entrapped near-surface enzyme), and the printed hydrogel retains >90% of its original activity following five hours of washing (FIG. 8). Furthermore, full substrate conversion occurred within 15 minutes for each entrapped enzyme, demonstrating that the thin printed hydrogel possesses a combination of sufficiently high porosity and low diffusional path length to allow for efficient diffusion of substrate molecules to the enzyme active sites and rapid read-out of enzyme activity. FIG. 8 shows a graph indicating that printing β-lactamase in a hydrogel minimizes enzyme leaching. Residual activity of samples washed for 5 h relative to the corresponding unwashed control is presented in the inset graph, confirming that minimal quantities of enzyme can be leached from the printed hydrogel. Error bars represent the standard deviation from the mean (n=3).

### Example 8: Protease Protection Studies

10 μL of a 2 mg/mL proteinase K solution (prepared in 10 mM PBS and 1 mM CaCl₂) was pipetted onto the printed enzyme samples both with and without hydrogel encapsulation. The samples were incubated in a closed container for 2 hours at room temperature, after which substrate solutions were pipetted onto the treated samples at the volumes listed in Table 1. Image acquisition and analysis was performed as described for the entrapment studies. The intensity of each sample was measured and presented as a ratio of the corresponding control image (untreated with protease). Table 1 shows the substrates and added volumes used for Alkaline phosphatase (AP), Urease and β-lactamase (β-Lac).

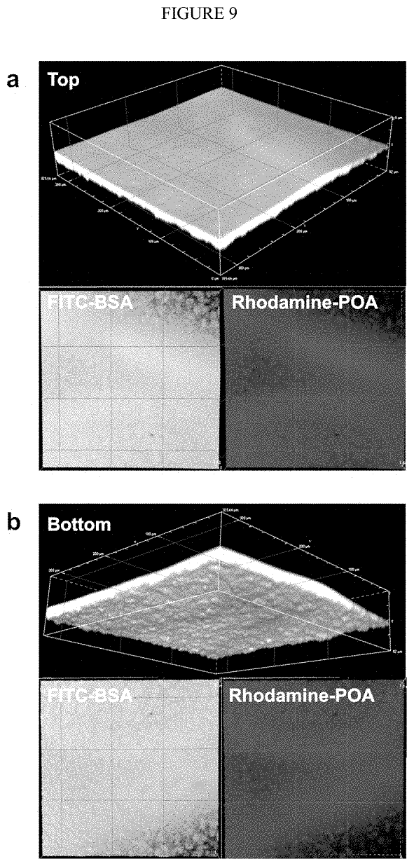

The printed hydrogel prevented proteolytic deactivation of all tested enzymes by proteinase K, with each enzyme retaining >80% of its pre-treatment activity (FIG. 9a-c); in contrast, urease and β-Lac printed directly on the nitrocellulose substrate without the hydrogel retained <10% of their activity over the same treatment time. While the steric barrier presented by the hydrogel is likely the main reason for this result, the ability of the PO-based hydrogel to resist non-specific protein adsorption may also be beneficial to reduce the probability of proteinase K binding close to the enzyme. AP was a slight outlier in this regard, retaining ˜40% of its activity when printed alone (consistent with its noted high stability relative to other enzymes)³⁵ and >80% of its activity when printed with POH even in the absence of gel formation (FIG. 9a). Co-printing enzymes with POH alone or in combination with unfunctionalized PO also showed limited benefits in terms of stabilizing both urease and β-Lac against proteolytic deactivation. However, the hydrogel-printed samples each demonstrated significantly better performance for all tested enzymes. FIG. 9 shows graphs indicating that the printed hydrogel protects enzyme (E) against proteolytic degradation and supports enzyme stabilization for long-term storage in one embodiment of the disclosure. Enzyme activity was quantified after 2 hours of protease treatment with proteinase K (a-c) or following long-term storage at room temperature (e-f) and normalized to the initial activity following printing. Enzymes: (a, d) Alkaline phosphatase (AP); (b, e) Urease; (c, f) β-lactamase (β-Lac). Error bars represent the standard deviation from the mean (n=3).

### Example 9: Long-Term Stability Studies

Printed enzyme samples were stored in a closed, dark container at room temperature for time periods ranging from 7 days up to 3 months. Image acquisition and analysis was performed as described previously for the entrapment and proteinase K degradation studies. The intensity of each sample was measured and presented as a ratio of the corresponding control image (freshly printed).

The hydrogel-entrapped enzymes retained ˜100% activity after at least three months of storage for AP, urease, and (β-Lac (FIG. 9d-f); in contrast, direct printed enzymes lost >70% of their activity within one week for urease and (β-Lac and within one month for AP. While similar efficacy in enzyme stabilization has previously been reported with dried carbohydrate films,³⁶ such films dissolve when placed back in an aqueous environment, leading to rapid leaching of the enzyme from the substrate. In contrast, the printed hydrogel maintains a confined environment for the enzyme under aqueous conditions (maintaining immobilization) while also maintaining local hydration to promote enzyme activity.

### Example 10: Cell Encapsulation

Mouse myoblast NIH 3T3 cells were pre-mixed at a density of 1×10⁶ cells/mL into the POH precursor polymer solution and hydrogels were printed as described above. Cells were pre-stained with CFSE stain such that they fluoresce green, and 3D images of the cell distribution within the hydrogels were collected using confocal microscopy. In another test, HepG2 cells were also pre-mixed inside a 8wt % PO10 gels at ˜700,000 cell s/mL and printed as described above. A LIVE/DEAD stain was then used to assess cell viability at different timepoints, with live cells fluorescing green and dead cells fluorescing red. Fluorescence imaging was conducted using a fluorescence plate reader with imaging capability.

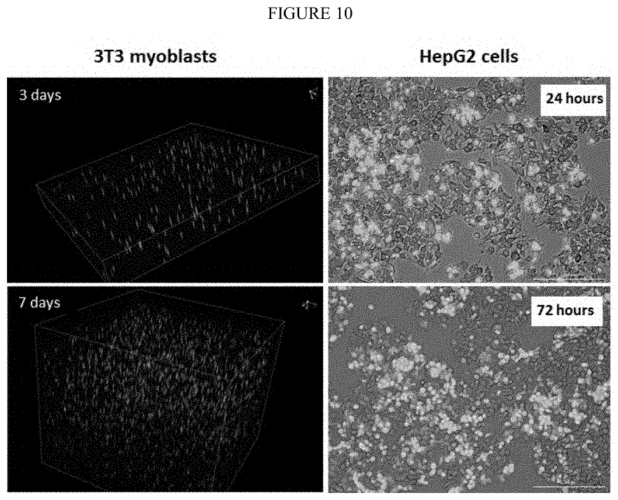

Confocal microscopy of the printed hydrogels indicates maintained 3T3 cell viability within the hydrogel over at least one week (FIG. 10, left), with increasing cell numbers also observed between 3 and 7 days indicating not only the presence of viable cells but also proliferating cells within the gel. LIVE/DEAD staining of the encapsulated HepG2 cells also shows high cell viability over multiple days (FIG. 10, right), with no dead cells visible in the images. Thus, the printable hydrogels can maintain high cell viability and, in some cases, promote cell proliferation.

### Example 11: Chaotropic Agent Denaturation Studies

10 μL of urea denaturation buffer (8 M urea, 5 mM dithiothreitol, 50 mM Tris-Cl (pH=7.5), 150 mM NaCl) was pipetted onto samples of 1 μM (β-lactamase entrapped in the printed hydrogel. The samples were incubated in a closed container for 30 min. at room temperature and then washed with DIW. Image acquisition and analysis was performed as described for the entrapment studies. The intensity of each sample was measured and presented as a ratio of the corresponding control image (samples treated with 10 mM PBS). For the solution denaturation study, 1 μM (β-lactamase was prepared in 100 μL of urea denaturation buffer and incubated for 30 min. at room temperature, after which nitrocefin was added to a final concentration of 200 (β-lactamase activity was then assessed via UV-vis spectrophotometry, tracking the hydrolysis of nitrocefin (Infinite M1000 spectrophotometer, Tecan) by monitoring solution absorbance at 492 nm. For the solution refolding study, 1 μM β-lactamase samples prepared in urea denaturation buffer were dialyzed against 10 mM PBS using a 3.5 kDa MWCO dialysis device (ThermoFisher) for 20 cycles (20 min/cycle). (β-lactamase activity was then re-assessed via UV-vis spectrophotometry as described above.

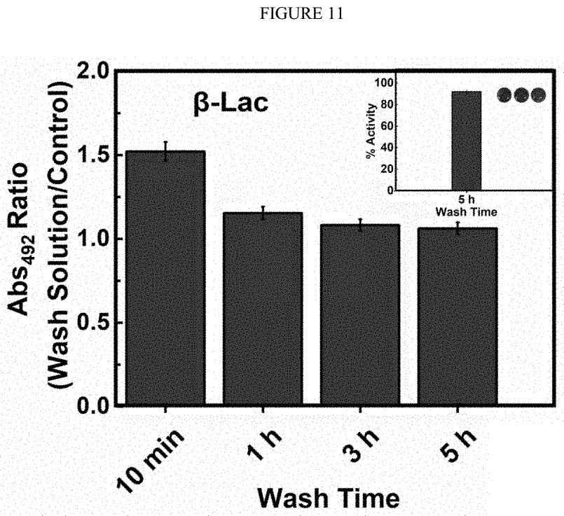

Printed hydrogels showed high efficacy in resisting chaotropic agent-induced denaturation, with hydrogel-printed β-Lac retaining >95% activity following urea challenge (similar to that observed following re-folding of the denatured protein via dialysis) (FIG. 11). In comparison, only <20% of protein activity was maintained when enzymes in solution were exposed to the same denaturation buffer. FIG. 10 shows a graph indicating that the printed hydrogel protects β-lactamase against chaotropic agent-induced denaturation. The remaining activity of β-lactamase printed in a hydrogel or in solution was quantified after 30 min. of treatment with urea denaturation buffer and normalized to the activity of the control incubated in 10 mM PBS. The solution refolding activity was measured by dialyzing a solution of β-lactamase prepared in urea denaturation buffer against 10 mM PBS in order to promote protein refolding and then re-testing the enzymatic activity. Error bars represent the standard deviation from the mean (n=3).

### Example 12: β-Lactamase Assay

Antibiotic resistance due to the β-lactamase mediated degradation of β-lactam antibiotics is a pressing issue, initiating widespread interest in discovering β-lactamase inhibitors in order to reclaim antibiotics that been previously rendered ineffective. In an embodiment of the invention, a high-throughput screening assay is developed as a drug screening platform for β-lactamase. The β-lactamase enzyme is printed in a printable hydrogel within the microzones of a wax printed 96-well nitrocellulose template. Inhibitor solutions and nitrocefin (a colorimetric β-lactamase substrate) are subsequently deposited onto the microzones at different concentrations using a high-throughput dispensing robot and the resulting colorimetric readout of β-lactamase activity is quantified via image analysis. The pore size of the printed hydrogel exercises size selectivity and is able to exclude promiscuous aggregating inhibitors from the encapsulated enzyme, correctly identifying the lack of activity of a variety of these compounds that give positive results in solution assays. Given that promiscuous inhibitors are arguably the most widespread artifact encountered in high-throughput screening, this technology demonstrates strong potential to streamline the drug discovery process by significantly reducing the number of false positive hits in early-stage lead identification.

### Solution-Based β-Lactamase Assay

True inhibitor (tazobactam, sulbactam and clavulanic acid) solutions were prepared in DIW and promiscuous inhibitor (rottlerin, BIS IX and TIPT) solutions were diluted in DIW from 10 mM DMSO stock solutions. The assay mixture contained 25 nM β-lactamase and a range of inhibitor concentrations (relevant to the IC₅₀ of the true inhibitors and the apparent IC₅₀ of the aggregating promiscuous inhibitors) in 100 μL of 10 mM PBS buffer. β-lactamase and inhibitor were pre-incubated for 10 minutes, after which nitrocefin was added to a final concentration of 200 μM. β-lactamase activity was then assessed via UV-vis spectrophotometry by tracking the hydrolysis of nitrocefin (Infinite M1000 spectrophotometer, Tecan) by monitoring solution absorbance at 492 nm.

### Printed Hydrogel-Based β-Lactamase Assay

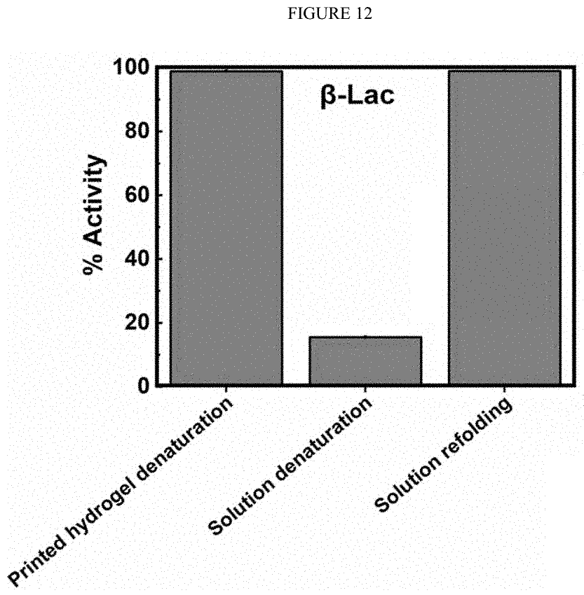

POH ink solution was prepared with a final concentration of 50 nM β-lactamase and used to print hydrogel spots on a 96-well paper microzone plate (as described previously, FIG. 12). 5 μL of tazobactam, sulbactam and clavulanic acid solutions (at starting concentrations of 100 μM) were added to each microzone using a Tecan Freedom Evo 200 liquid handling robot (Tecan, Switzerland). The inhibitor was incubated with the printed β-lactamase for 20 minutes, after which the assay was initiated with the addition of 5 μL of nitrocefin (500 μM) to each microzone. A similar protocol was used to test the promiscuous inhibitors rottlerin, BIS IX and TIPT, again using starting concentrations of 100 μM. Images were taken with a Canon DSLR camera (operated in manual focus mode without flash) after 25 min. The wax printed background was removed in GIMP software (Version 2.8.16). Image analysis was performed using Fiji, with the converted substrate colour extracted using the Color Deconvolution plugin. Extracted images were inverted and converted to 8 bit grayscale images. The intensity of each sample was measured and presented as a ratio of the control image (not treated with inhibitor). Calculation of IC₅₀ values was carried out in OriginPro by plotting the calculated percentage inhibition against the added inhibitor concentration. Curve fitting was performed with the dose-response function (OriginLab Corporation, Northampton, Mass. U.S.A.). FIG. 12 shows the printable hydrogel microarray for drug screening in one embodiment of the disclosure. Hydrogel spots entrapping β-lactamase were printed on microzones that are created by wax-printing a 96-well pattern on nitrocellulose.

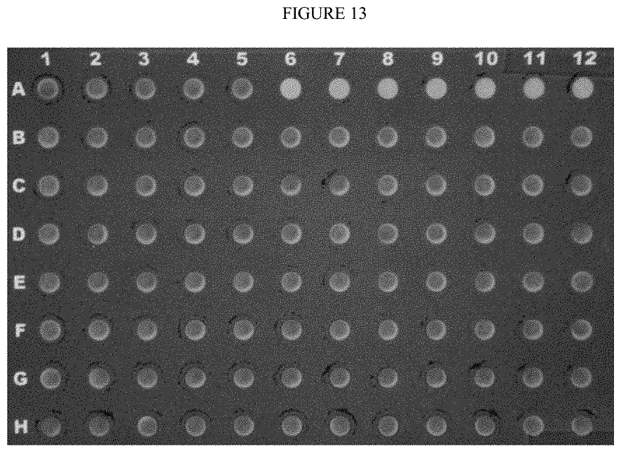

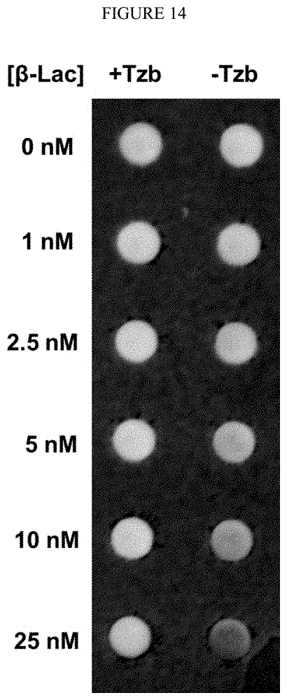

Using the printed hydrogel assay, IC₅₀ values of 0.071 μM, 4.1 μM and 0.15 μM were calculated for tazobactam, sulbactam and clavulanic acid respectively (FIG. 13a-c, Table 2); these values compare favorably to the measured solution-based assay IC₅₀ values (FIG. 13a-c, Table 2) as well as literature IC₅₀ values (Table 2) for these same inhibitors but require only 10% of the total sample volume, a significant benefit in screening high value potential inhibitors. Significant and detectable colour differences were observed using enzyme concentrations as low as 5 nM, providing additional flexibility for the assay in detecting low K_{I} inhibitors (FIG. 14). FIG. 13 shows graphs indicating that printed hydrogel-based β-lactamase screening assay can determine dose-response relationships of classic β-lactamase inhibitors and discriminate between true and promiscuous aggregating inhibitors in one embodiment of the disclosure: (a-c) Comparison of solution versus printed hydrogel-based inhibition curves for true β-lactamase inhibitors: (a) tazobactam; (b) sulbactam; (c) clavulanic acid. (d-f) Comparison of solution versus printed hydrogel-based inhibition curves for known promiscuous inhibitors of β-lactamase: (d) rottlerin; (e) BIS IX; (f) TIPT. Error bars represent the standard deviation from the mean (n=3). FIG. 13 shows the selection of the optimal β-lactamase concentration in a printed hydrogel-based screening assay in one embodiment of the disclosure. A range of β-lactamase concentrations was printed in the hydrogel, with the colorimetric readouts compared with and without tazobactam (100 μM) treatment. Table 2 shows the comparison of IC₅₀ values of classic β-lactamase inhibitors measured by the printed hydrogel assay relative to the conventional solution assay and reported literature values. Error represents the standard deviation from the average of three replicate assays. Note: literature values reported from Payne et al (1994).³⁴

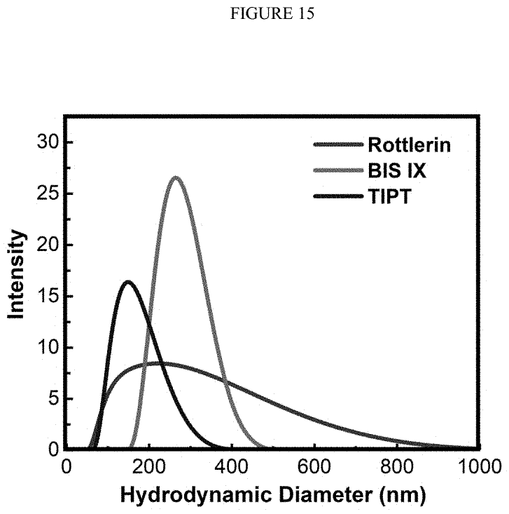

The quantitative correlation between these results suggests that the printed hydrogel-based assay can determine dose-response relationships of β-lactamase inhibitors with high accuracy. Following, to assess the capacity of the printed hydrogels to differentiate between specific and non-specific inhibition, the confirmed promiscuous inhibitors rottlerin and BIS IX (both kinase inhibitors) and tetraiodophenolphthalein (TIPT, another established aggregate forming compound) were tested against TEM-1 β-lactamase (an isoform of β-lactamase) both in solution (modeling a conventional microplate assay) and using a printed hydrogel array. In each case, the aggregating compounds inhibited β-lactamase in the solution-based assay (a false positive hit) but were correctly observed to induce no specific inhibition of β-lactamase in the hydrogel-based assay (FIG. 13d-f). Comparing the aggregate diameter range of ˜154-365 nm (Table 3, FIG. 15) to the characteristic correlation length (i.e. average pore size) of 20 Å⁻¹ for PO hydrogels of this type, without wishing to be bound by theory, we hypothesize that the aggregates cannot diffuse into the hydrogel and thus are unable to sterically inhibit the enzyme. In this way, the size selectivity of the printed hydrogel layer excludes the promiscuous inhibitors from accessing the enzyme encapsulated in the hydrogel and thus avoids the false positive hits observed in solution assays. FIG. 15 shows a graph indicating the particle size distribution of promiscuous aggregating inhibitors (Rottlerin, BIS IX, TIPT). Intensity distributions are measured using dynamic light scattering (normalized to maximum intensity). Table 3 shows the aggregate size and polydispersity of 100 μM solutions of Rottlerin, BIS IX and TIPT, measured using dynamic light scattering (DLS).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| | | Volume of Substrate |
|---|---|---|
| Enzyme | Substrate | Added (μL) |
| Alkaline phosphatase | BCIP ®/NBT-Purple | 10 |
| (AP) | Liquid Substrate System | |
| Urease | 0.5 mM acetic acid, | 20 |
| | 5 mM urea, 0.005% | |
| | phenol red | |
| β-lactamase | 500 μM nitrocefin | 10 |
| (β-Lac) | (19.4 μM DMSO stocks | |
| | diluted in 10 mM PBS) | |

TABLE 2

IC₅₀ (μM)

| β-Lactamase | Printed hydrogel | Solution | |
|---|---|---|---|
| Inhibitor | assay | assay | Literature |
| Tazobactam | 0.07 ± 0.01 | 0.06 ± 0.01 | 0.04 |
| Sulbactam | 4.1 ± 0.2 | 4.0 ± 0.3 | 6.1 |
| Clavulanic acid | 0.15 ± 0.01 | 0.19 ± 0.01 | 0.09 |

TABLE 3

| Inhibitor | Size (nm) | Polydispersity |
|---|---|---|
| Rottierin | 188 ± 1 | 0.24 ± 0.01 |
| BISIX | 365 ± 9 | 0.36 ± 0.02 |
| TIPT | 154 ± 1 | 0.16 ± 0.02 |

### FULL CITATIONS FOR DOCUMENTS REFERRED TO IN THE APPLICATION

1. Zhao, X., Qi, F., Yuan, C., Du, W. & Liu, D. Lipase-catalyzed process for biodiesel production: Enzyme immobilization, process simulation and optimization. Renewable and Sustainable Energy Reviews 44, 182-197 (2015).

2. Alkasir, R. S., Ornatska, M. & Andreescu, S. Colorimetric paper bioassay for the detection of phenolic compounds. Analytical Chemistry 84, 9729-9737 (2012).

3. Xue, P., Gu, Y., Su, W., Shuai, H. & Wang, J. In situ one-pot preparation of superparamagnetic hydrophilic porous microspheres for covalently immobilizing penicillin G acylase to synthesize amoxicillin. Applied Surface Science 362, 427-433 (2016).

4. Forsberg, E. M., Sicard, C. & Brennan, J. D. Solid-Phase Biological Assays for Drug Discovery. Annual Review of Analytical Chemistry 7, 337-359 (2014).

5. Arenkov, P. et al. Protein Microchips: Use for Immunoassay and Enzymatic Reactions. Analytical Biochemistry 278, 123-131 (2000).

6. Küchler, A., Yoshimoto, M., LuginbUhl, S., Mavelli, F. & Walde, P. Enzymatic reactions in confined environments. Nature Nanotechnology 11, 409-420 (2016).

7. Wang, Q., Yang, Z., Wang, L., Ma, M. & Xu, B. Molecular hydrogel-immobilized enzymes exhibit superactivity and high stability in organic solvents. Chemical Communications, 1032-1034 (2007).

8. Mariani, A. M., Natoli, M. E. & Kofinas, P. Enzymatic activity preservation and protection through entrapment within degradable hydrogels. Biotechnology and Bioengineering 110, 2994-3002 (2013).

9. Kutcherlapati, S. R., Yeole, N. & Jana, T. Urease immobilized polymer hydrogel: Long-term stability and enhancement of enzymatic activity. Journal of Colloid and Interface Science 463, 164-172 (2016).

10. Qian, Y.-C., Chen, P.-C., Zhu, X.-Y. & Huang, X.-J. Click synthesis of ionic strength-responsive polyphosphazene hydrogel for reversible binding of enzymes. RSC Advances 5, 44031-44040 (2015).

11. Yoshimura, I. et al. Molecular recognition in a supramolecular hydrogel to afford a semi-wet sensor chip. Journal of the American Chemical Society 126, 12204-12205 (2004).

12. Yang, Q., Adrus, N., Tomicki, F. & Ulbricht, M. Composites of functional polymeric hydrogels and porous membranes. Journal of Materials Chemistry 21, 2783-2811 (2011).

13. Ikeda, M., Ochi, R. & Hamachi, I. Supramolecular hydrogel-based protein and chemosensor array. Lab on a Chip 10, 3325-3334 (2010).

14. Lilly, J. L., Romero, G., Xu, W., Shin, H. Y. & Berron, B. J. Characterization of molecular transport in ultrathin hydrogel coatings for cellular immunoprotection. Biomacromolecules 16, 541-549 (2015).

15. Deng, X. et al. Poly (oligoethylene glycol methacrylate) Dip-Coating: Turning Cellulose Paper into a Protein-Repellent Platform for Biosensors. Journal of the American Chemical Society 136, 12852-12855 (2014).

16. Shundo, A. et al. Facile microcapsule fabrication by spray deposition of a supramolecular hydrogel. RSC Advances 4, 36097-36100 (2014).

17. Chollet, B. et al. Multiscale Surface-Attached Hydrogel Thin Films with Tailored Architecture. ACS Applied Materials & Interfaces 8, 11729-11738 (2016).

18. Limem, S., McCallum, D., Wallace, G. G. & Calvert, P. Inkjet printing of self-assembling polyelectrolyte hydrogels. Soft Matter 7, 3818-3826 (2011).

19. De Gans, B.-J. & Schubert, U.S. Inkjet printing of well-defined polymer dots and arrays. Langmuir 20, 7789-7793 (2004).

20. Andres, C. M. & Kotov, N. A. Inkjet deposition of layer-by-layer assembled films. Journal of the American Chemical Society 132, 14496-14502 (2010).

21. Derby, B. Inkjet printing of functional and structural materials: fluid property requirements, feature stability, and resolution. Annual Review of Materials Research 40, 395-414 (2010).

22. Chen, G. Y., Uttamchandani, M., Zhu, Q., Wang, G. & Yao, S. Q. Developing a Strategy for Activity-Based Detection of Enzymes in a Protein Microarray. ChemBioChem 4, 336-339 (2003).

23. Mitchell, P. A perspective on protein microarrays. Nature Biotechnology 20, 225-229 (2002).

24. Shoichet, B. K. Screening in a spirit haunted world. Drug Discovery Today 11, 607-615 (2006).

25. Rishton, G. M. Nonleadlikeness and leadlikeness in biochemical screening. Drug Discovery Today 8, 86-96 (2003).

26. McGovern, S. L., Helfand, B. T., Feng, B. & Shoichet, B. K. A specific mechanism of nonspecific inhibition. Journal of Medicinal Chemistry 46, 4265-4272 (2003).

27. McGovern, S. L. & Shoichet, B. K. Kinase inhibitors: not just for kinases anymore. Journal of Medicinal Chemistry 46, 1478-1483 (2003).

28. Seidler, J., McGovern, S. L., Doman, T. N. & Shoichet, B. K. Identification and prediction of promiscuous aggregating inhibitors among known drugs. Journal of Medicinal Chemistry 46, 4477-4486 (2003).

29. Duan, D., Doak, A. K., Nedyalkova, L. & Shoichet, B. K. Colloidal aggregation and the in vitro activity of traditional Chinese medicines. ACS Chemical Biology 10, 978-988 (2015).

30. Ryan, A. J., Gray, N. M., Lowe, P. N. & Chung, C.-w. Effect of detergent on “promiscuous” inhibitors. Journal of Medicinal Chemistry 46, 3448-3451 (2003).

31. Irwin, J. J. et al. An aggregation advisor for ligand discovery. Journal of Medicinal Chemistry 58, 7076-7087 (2015).

32. Feng, B. Y. & Shoichet, B. K. A detergent-based assay for the detection of promiscuous inhibitors. Nature Protocols 1, 550-553 (2006).

33. Ferreira, R. S. et al. Complementarity between a docking and a high-throughput screen in discovering new cruzain inhibitors. Journal of Medicinal Chemistry 53, 4891-4905 (2010).

33a. https://www. sciencedirect.com/science/article/pii/S0142961208002871

34. Payne, D. J. et al. Comparative activities of clavulanic acid, sulbactam, and tazobactam against clinically important beta-lactamases. Antimicrobial Agents and Chemotherapy 38, 767-772 (1994).

35. McComb, R. B., Bowers Jr, G. N. & Posen, S. Alkaline Phosphatase, (Springer Science & Business Media, 2013).

36. Cicerone, M. T. & Soles, C. L. Fast dynamics and stabilization of proteins: binary glasses of trehalose and glycerol. Biophysical Journal 86, 3836-3845 (2004).

1. A hydrogel that:
a) forms a gel on a substrate from precursor polymer building block(s);
b) can immobilize a bioactive biomolecule and/or cell and;
c) can control access to that biomolecule and/or cell by other chemicals in the hydrogel environment.

2. The hydrogel as claimed in claim 1, wherein said hydrogel is in situ gelling.

3. The hydrogel as claimed in claim 1, wherein said hydrogel is printable.

4. The hydrogel as claimed in claim 1, comprising poly(ethylene glycol), poly(oligoethylene glycol acrylate), poly(oligoethylene glycol methacrylate), poly(sulfobetaine), poly(carboxybetaine), or derivatives thereof

5. The hydrogel as claimed in claim 4, formed by mixing two covalently crosslinkable functionalized pre-polymers.

6. The hydrogel as claimed in claim 5, formed by sequential printing of the two covalently crosslinkable functionalized pre-polymers.

7. The hydrogel as claimed in claim 6, crosslinked by hydrazone bonds.

8. The hydrogel as claimed in claim 1, formed using sequential printing of aldehyde-functionalized poly(oligoethylene glycol methacrylate) and hydrazide-functionalized poly(oligoethylene glycol methacrylate).

9. A hydrogel of the type described in claim 1, wherein the substrate comprises cellulose, nitrocellulose, cellulose acetate, glass, polysulfone, polyacrylonitrile, polystyrene, polypropylene, or polyethylene.

10. A hydrogel of the type described in claim 1, wherein the hydrogel is printed in a microarray format, the printed microarray format can be incorporated into conventional high-throughput screening assays.

11. A hydrogel of the type described in claim 1, wherein the bioactive biomolecule is a cell, protein, enzyme, DNA, RNA, aptamer, other polynucleotide, carbohydrate, proteoglycan, or glycoprotein.

12. A method for a screening drug candidate against a bioactive biomolecule and/or cell, the method comprising
a) printing a hydrogel on a substrate, wherein the hydrogel is embedded with a bioactive biomolecule and/or cell;
b) depositing a solution of a drug candidate and an analyte specific to the biomolecule and/or cell on the hydrogel;
c) quantitatively assessing the activity of the drug candidate on the biomolecule and/or cell.

13. The method of claim 12, wherein the hydrogel is printed in a microarray format.

14. The method of claim 13, wherein the printed microarray format can be incorporated into conventional high-throughput screening assays.

15. The method of claim 12, wherein the bioactive biomolecule is a protein, enzyme, DNA, RNA, aptamer, other polynucleotide, carbohydrate, proteoglycan, or glycoprotein.

16. The method of claim 15, wherein the enzyme is β-lactamase.

17. The method of claim 12, wherein the hydrogel comprises,
a) at least one first precursor polymer which is a hydrazide-functionalized poly(oligoethylene glycol methacrylate) copolymer, and
b) a second precursor polymer which is an aldehyde- and/or ketone-functionalized poly(oligoethylene glycol methacrylate) copolymer,
wherein the first and second precursor polymers are crosslinked through hydrazone bonds to form the hydrogel.

18. The method of claim 17, wherein the hydrogel is formed by sequential printing of the first precursor polymer and the second precursor polymer.

19. A drug screening platform, comprising:
a) a substrate;
b) a hydrogel printed on the substrate; and
c) a biomolecule and/or cell entrapped in the hydrogel.

20. The drug screening platform of claim 19, wherein the hydrogel comprises,
a) at least one first precursor polymer which is a hydrazide-functionalized poly(oligoethylene glycol methacrylate) copolymer, and
b) a second precursor polymer which is an aldehyde- and/or ketone-functionalized poly(oligoethylene glycol methacrylate) copolymer, wherein the first and second precursor polymers are crosslinked through hydrazone bonds to form the hydrogel.
